(19) United States
(12) Patent Application Publication
(10) Pub. No.: US 2015/0202090 A1
(43) Pub. Date: Jul. 23, 2015

(54) Absorbent Article

(71) Applicant: Renew Medical, Inc., Palo Alto, CA (US)

(72) Inventors: Tidhar Shalon, Palo Alto, CA (US); Lisa Carmel, Mill Valley, CA (US); Ajit Shah, Portola Valley, CA (US)

(73) Assignee: Renew Medical, Inc., Palo Alto, CA (US)

(21) Appl. No.: 14/423,707

(22) PCT Filed: Sep. 6, 2013
(86) PCT No.: PCT/US2013/058487

§ 371 Date: Feb. 24, 2015

Related U.S. Application Data
(60) Provisional application No. 61/698,223, filed on Sep. 7, 2012.

(51) Int. Cl. A61F 13/15; A61F 13/66

(52) U.S. Cl. CPC A61F 13/15; A61F 13/66; A61F 2013/1513

(57) ABSTRACT

An incontinence pad is provided. The device includes an anal region and optionally a genital region.

### CROSS-REFERENCE TO RELATED APPLICATIONS

This application is a national phase application of the PCT international application number PCT/US2013/058487 titled “Absorbent Article”, filed in the United States Patent and Trademark Office as the Receiving Office on Sep. 6, 2013, which claims priority to and the benefit of provisional patent application No. 61/698,223 titled “Absorbent Article”, filed in the United States Patent and Trademark Office on Sep. 7, 2012. The specifications of the above referenced patent applications are incorporated herein by reference in their entirety.

### BACKGROUND OF THE INVENTION

### Field of the Invention

The present invention relates to absorbent article that includes a bowel incontinence pad, either alone or in combination with a pad for urinary incontinence or menses.

Bowel incontinence (also referred to as fecal, anal or rectal incontinence, or accidental bowel leakage) is a disorder afflicting both children and adults with devastating social and psychological affects. It is estimated that in the United States alone more than 5.5 million individuals suffer from bowel incontinence and that the incidence rate of this disorder is 1-5%. Bowel incontinence is a major factor limiting the rehabilitation of the elderly and disabled, preventing many of them from being cared for at home. Mild to moderate incontinence accounts for about 80% of the market (gas, liquid and soiling) while severe incontinence accounts for the remaining 20%.

Bowel continence results from a coordinated motor function of anal sphincters and pelvic floor muscles, the role of the rectum and sigmoid colon as a bowel reservoir with capacitance and compliance and as a propulsive force with intrinsic motor activity, the effects of stool consistency, volume and delivery rate, the anorectal angle, and anorectal sensation.

The most common causes of bowel incontinence are structural or functional deficiencies of the sphincter muscles. Such deficiencies can result from anatomic disruption of the sphincter mechanism which may be caused by obstetric injuries (perineal laceration and improperly performed median episiotomies), complications of hemorrhoid, fistula or fissure surgery (keyhole deformities), traumatic injuries (e.g., impalement injuries), or cancer or from deterioration of the sphincter muscles due to age, congenital disorders, systemic and metabolic diseases, acquired neurological defects, and diseases of the colon and rectum.

Present treatment approaches for restoring bowel continence include non-surgical and surgical therapy. Non-surgical therapy for incontinence include anal plugs or anal inserts, biofeedback and perianal strengthening exercises beneficial in alleviating symptoms of seepage and occasional loss of control and electrical stimulation to improve contraction of the sphincter muscles.

Surgical therapy approaches include implantation of artificial valves (see, for example, U.S. Pat. Nos. 6,471,635, 6,749,556, and U.S. patent application Ser. Nos. 10/269,949 and 10/651,851), injection of bulking agents into the anal mucosa or the anal sphincters (see, for example, Vaizey and Kamm, British Journal of Surgery 2005; 92: 521-527), implanted electrodes for stimulating the pudendal or sacral nerves (see, for example, U.S. Pat. Nos. 6,907,293 and 7,054,689) or sphincteric muscles (see, for example, PCT publication No. WO06047833).

Many sufferers of bowel incontinence do not desire any of the above treatment options, but instead choose to cope with BI using undergarment protection such as pads or diapers. However, absorbent pads or diapers designed for managing bowel incontinence still leave bowel discharge in contact with external tissue causing major irritation of the perianal area and buttocks cheeks (e.g. diaper rash). Furthermore, presently known pads or diapers allow bowel fluids to leak or wick to the vaginal areas in women (causing infections), and are not optimized to catch or absorb just small amounts of leakage of liquid, mucosal or solid stool, which is the case for the majority of the light to moderate bowel incontinence sufferers. Lastly, pads used for BI do not control odor as the discharge does not come into contact with the pad until sufficient volume exists to travel down the length of the buttocks cheek to the pad itself.

Thus, there remains a need for a bowel incontinence pad that overcomes the aforementioned limitations of known pads and diapers.

### BRIEF SUMMARY OF THE INVENTION

According to one aspect of the present invention, an absorbent article comprising an anal pad is configured for folding along an axial center line to fit between buttocks cheeks, and further configured for wicking moisture away from an anus to an absorbent material positioned against said buttock cheeks and/or at a genital region.

According to a further feature in preferred embodiments of the invention described below, the anal pad includes a first pad region for contacting an anus and a second pad region with an absorbent material.

According to further features in preferred embodiments of the invention described below, the anal pad is held in position up against the anus using adhesive and/or a ventral strap.

According to further features in preferred embodiments of the invention described below, there is provided an absorbent article having an anal pad and a genital region pad not being in fluid communication with one another.

According to further features in preferred embodiments of the invention described below, the anal pad is configured for occupying a gluteal cleft space and it comprises a central ridge at least 2 cm higher than the perimeter of the pad when the article is worn by a user.

According to still further features in the described preferred embodiment the anal pad overlaps at least some region of the genital region pad.

According to still further features in the described preferred embodiment the anal pad can assume a complex curve with maximum radius of 10 cm in at least two dimensions simultaneously without losing conformance to the anatomy.

According to still further features in the described preferred embodiment the anal pad includes a bowel discharge collection pouch.

According to still further features in the described preferred embodiment the anal pad includes a pair of folded buttock cheek pads.

According to still further features in the described preferred embodiment the bowel discharge collection pouch applies up to 100 grams of force to open up the buttock cheek pads in the lateral direction.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates a bowel incontinence (BI) pad constructed in accordance with the teachings of the present invention.

FIG. 2. illustrates a strapped BI pad in combination with a female genital pad constructed in accordance with the teachings of the present invention.

FIG. 3 illustrates a strapped BI pad in combination with a male genital pouch constructed in accordance with the teachings of the present invention.

FIG. 4 illustrates a BI pad in combination with a front genital opening constructed in accordance with the teachings of the present invention.

FIGS. 5A-E illustrate a BI pad with an anal opening and a bowel discharge collection pouch constructed in accordance with the teachings of the present invention.

FIGS. 6A-C illustrate a BI pad attachable to a waistband or underwear of a user constructed in accordance with the teachings of the present invention.

FIG. 7 illustrates a BI pad with an anal penetrating element and a skin adhesive region constructed in accordance with the teachings of the present invention.

FIG. 8 illustrates a BI pad whose buttock cheek pads can move or rotate with respect to one another constructed in accordance with the teachings of the present invention.

FIGS. 9A-C illustrate an anal region of a BI pad with various embodiments of an anal penetrating element constructed in accordance with the teachings of the present invention.

FIGS. 10A-B illustrate a prior art combined urinary incontinence (UI) and bowel incontinence (BI) pad (FIG. 10A) and a UI/BI pad constructed in accordance with the teachings of the present invention (FIG. 10B) superimposed on a midline sagittal section of the female pelvic anatomy.

FIGS. 11A-D illustrate a combined UI/BI pad constructed in accordance with the teachings of the present invention from 4 different angles.

FIG. 12 illustrates a combined UI/BI pad with an anal opening and a bowel discharge pouch constructed in accordance with the teachings of the present invention.

FIG. 13 illustrates a stick-on BI pad with an anal opening and a bowel discharge pouch constructed in accordance with the teachings of the present invention.

FIG. 14 illustrates a stick-on BI pad with an anal opening, an anal penetrating element and a bowel discharge pouch constructed in accordance with the teachings of the present invention.

FIG. 15 illustrates a combined UI/BI pad where a region of the two pads overlap each other.

FIGS. 16A-D illustrate a protective finger or hand cot onto which is mounted an absorbent article.

### DETAILED DESCRIPTION OF THE INVENTION

The present invention is an absorbent article or pad (with or without a collection pouch) that can be used to treat mild to moderate bowel incontinence consisting of small amounts of liquid, mucosal or solid stool. The terms absorbent article and pad are used interchangeably herein. The pad captures BI discharges either alone, or in combination with urinary incontinence or menses. The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Bowel incontinence is a socially devastating disorder which affects at least 2.2 percent of community dwelling adults and 45 percent of nursing home residents. People who have bowel incontinence may feel ashamed, embarrassed, or humiliated; some don't want to leave the house out of fear they might have an accident in public. Treatment of bowel incontinence depends on the cause and severity of the disorder.

Severe cases are typically treated by surgeries for repairing damaged sphincters, reinforcing anorectal structures, implanting artificial sphincters, and transferring muscle tissue. Mild to moderate cases of bowel incontinence are typically treated using special diets, medication, bowel training, anal inserts or diapers. Although diapers can help contain bowel discharge, they are either ineffective or are unacceptable for many patients due to skin breakdown, vaginal contamination or odor issues. Thus, many patients remain unsatisfied with the design of existing pads and diapers for dealing with their condition.

From original market research among sufferers of bowel incontinence conducted by the inventors, it emerged that the majority of sufferers only leak small amounts of liquid, mucosal or solid stool. For this group of people, the all-encompassing heavy-duty incontinence diapers are too stigmatizing, cumbersome and bulky for their needs. These people usually resort to using feminine hygiene pads (such as used for urinary incontinence or menstruation) to protect them from accidental bowel leakage.

A limitation of current pads or diapers is that they follow the surface taken by underwear either due to being too wide, too stiff to fold along their midlines, or because they are attached to the underwear with an adhesive strip. As a result, current pads don't enter into the gluteal cleft and can't reach the anus where the discharge occurs. The curve adopted by the prior art pads is generally concave up in the dorsal/ventral axis and also slightly concave up along the curve of the genitals or buttocks, similar to a swath along the surface of a sphere with both curvatures pointing towards the inside of the sphere.

A study conducted by the present inventors among BI sufferers using prior art pads revealed that bowel discharge remain up in the gluteal cleft or travels down the buttocks cheeks before coming into contact with the undergarment protection pad. Bowel discharge fluids left in contact with the perianal area and buttocks cheek skin causes major skin irritation (“diaper rash”) and leads to release of embarrassing odors while also potentially contaminating the vaginal area.

Even those users who used the B-Sure™ butterfly-shaped anal incontinence pads (Birchwood Laboratories, Inc. Eden Prairie, Minn.) that fit into the gluteal cleft complained that such pads did not stay in the gluteal cleft and therefore bowel leakage still irritated the perianal and buttock cheek areas.

The present inventors have discovered that a BI pad that answers the needs of the light to moderate BI patient population should have the following attributes:

i. remain adjacent to the anus while standing, walking and sitting

ii. be comfortable to the point of almost imperceptible

iii. discreet (i.e. no visible diaper line or making of sound when walking)

iv. absorb bowel leakage as soon as it is discharged from the anus

v. in cases of solid leakage, retain the solid stool in a collection pouch to keep it out of contact with perianal and buttocks tissue

vi. to trap odor with active agents in the pad

vii. to not allow bowel discharges to wick or leak to the genital area to prevent infections.

While reducing the present invention to practice, the present inventors devised a BI pad having a first curvature along an axis that matches the dorsal-ventral curve (with a maximum radius of approximately 10 cm) and a second curvature in the opposite direction and along an axis that matches the gluteal cleft curve defined by the surfaces of the upper buttock cheeks (with a maximum radius of approximately 10 cm). In this manner, the anal pad can assume a complex curve with maximum radius of 10 cm along at least two axes simultaneously without losing conformance to the anatomy.

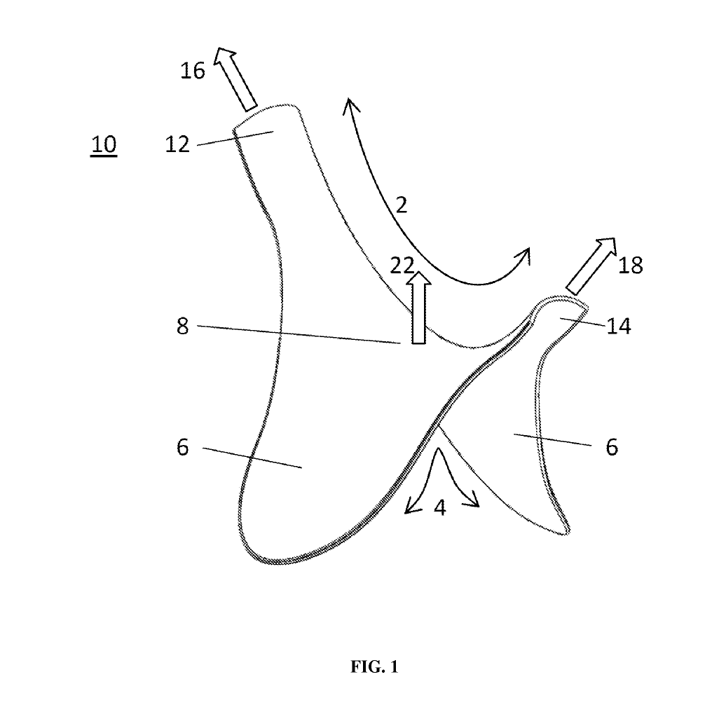

Referring now to the drawings, FIGS. 1-14 illustrate a BI hygienic article (or BI pad or absorbent article), either alone or in combination with a menses or urinary incontinence (UI) pad. The BI pad can optionally incorporate an anal penetrating element to help block the flow of feces, or to wick or channel away liquid stool to an absorbent reservoir or collection pouch.

Thus, according to one aspect of the present invention and as is illustrated in FIG. 1, there is provided a bowel incontinence (BI) pad 10 that along one axis that can easily conform to ventral-dorsal curve 2. BI pad 10 has two buttock cheek pads 6 that can dynamically adjust to gluteal cleft curve 4, thereby conforming to the complex curve surface defined by curves 2 and 4, and form a saddle-shaped pad that stays in close proximity to the anus and absorbs any bowel discharges that reach the perianal area and buttock cheek skin.

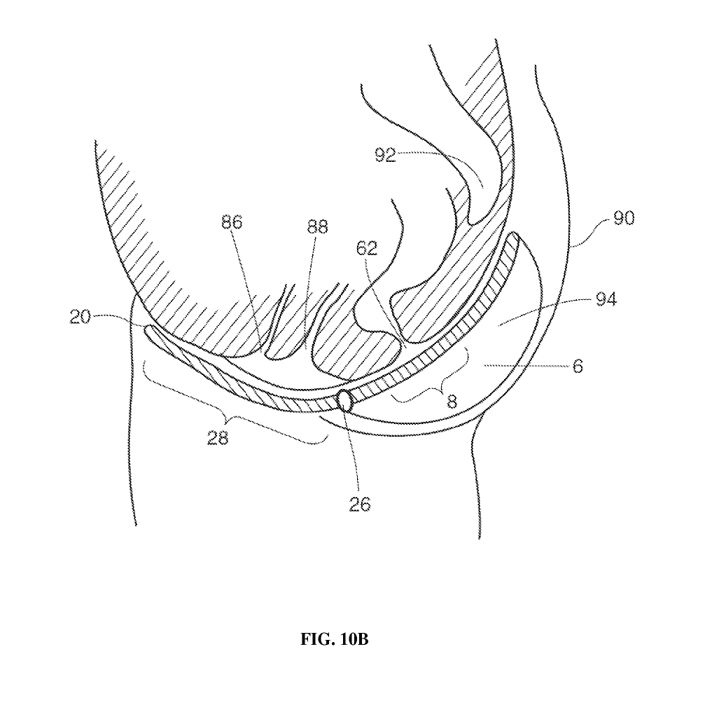

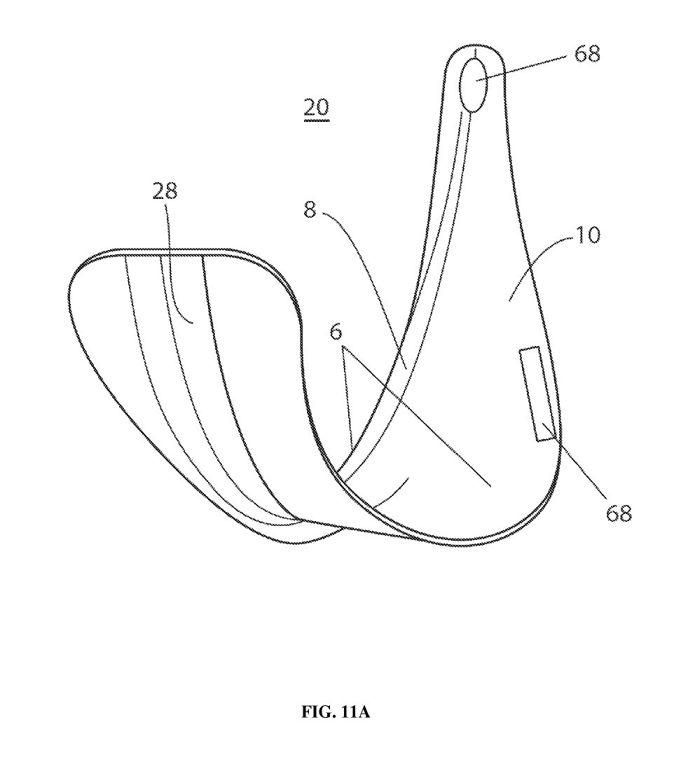

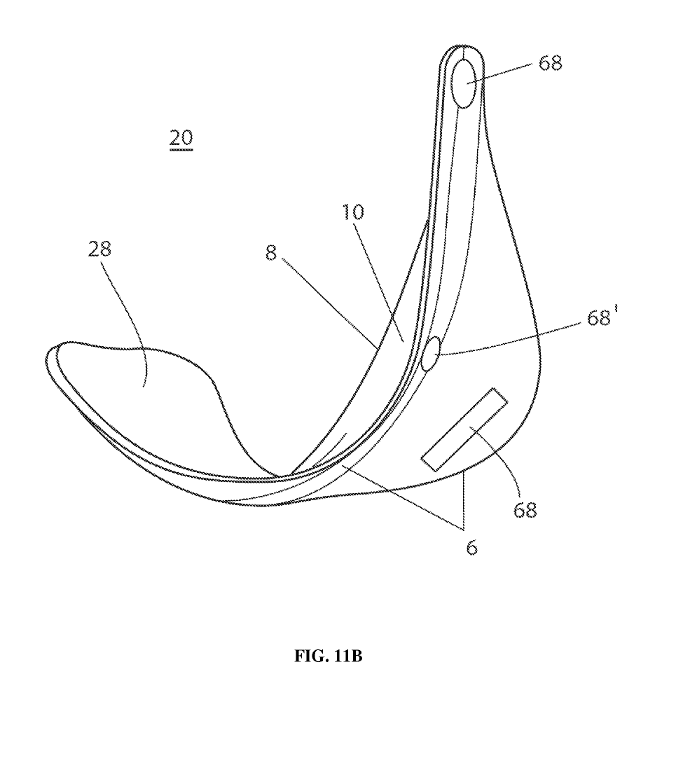

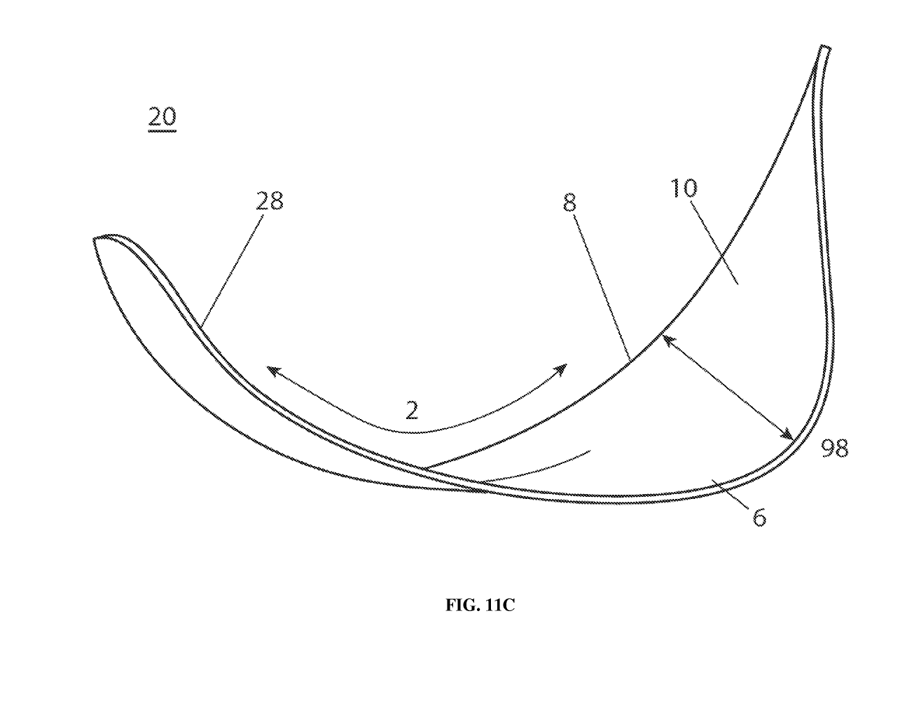

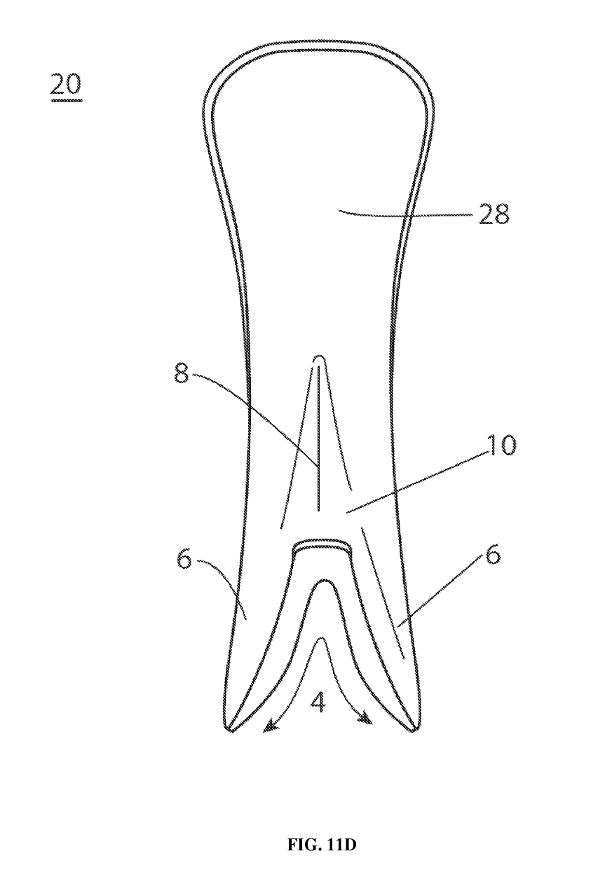

FIGS. 10A-B further illustrate the difference between prior art pads and the absorbent article of the current invention shown in section view superimposed on a sagittal section of the female pelvic anatomy. In FIG. 10A, prior art pad 96 has a rather homogenous elliptical or dog-bone shaped plan view and is up to 30-45 cm long and approximately 5-8 cm wide. Prior art pad 96 has genital region pad 28 which is intended to collect menses from vagina 88 and urine from urethra 86, as well as anal region 8 which is intended to absorb bowel discharge from anus 62. However, prior art pad 96 is of uniform thickness and width and requires at least 50-100 grams of force to fold back on itself along the longest midline of the pad. As a result, anal region 8 rides together with the underwear along the external surface of buttocks 90. Indeed, most prior art pads are adhered to the underwear in anal region 8 with adhesive and so are constrained and prevented from entering into the gluteal cleft along curve 4 (see FIGS. 1 and 11D). Therefore, any bowel discharge from anus 62 has to travel several centimeters along buttock cheek 94 before it reaches prior art pad 96. The perianal region and buttock cheek 94 tend to get easily irritated when exposed to stool, causing significant discomfort. Furthermore, the leaked stool in buttock cheek 94 cannot be neutralized for odor as it is not in contact with the pad. Furthermore, versions of prior art pads 96 that incorporate a bowel discharge pouch are ineffective as the fecal matter spreads out between buttock cheeks 94 and is not easy to collect at any one point along pad 96. Collection needs to occur adjacent to anus 62 and not at the surface of buttocks 90. Full incontinence diapers (similar to baby diapers) suffer from the same disadvantages as prior art pad 96 in that the rear portion of the diaper rides along the external surface of buttocks 90.

In sharp contrast, FIG. 10B illustrates pad 20 of the present invention where anal region 8 is pre-shaped according to curve 4 (see FIGS. 1 and 11D) or has folds 24 or is otherwise thin and elastic enough to fold on itself along the midline with less than 50 grams of force, preferably less than 30 grams of force. Therefore, anal region 8 folds in the shape of curve 4 and rides up along the top of the gluteal cleft adjacent to anus 62 towards tail bone 92. Leakage from anus 62 is collected, wicked or absorbed before it can contact buttock cheek 94 (hidden behind buttock cheek pad 6). In addition, barrier 26 prevents bowel and urine/menses fluids from intermixing.

FIGS. 11A-D illustrate perspective views of pad 20 when in the worn configuration. When packaged, pad 20 can be either flat, or have a preset shape of the worn configuration, yet remain stackable for efficient packaging (like Pringles™ potato chips). FIG. 11A illustrates a perspective view of pad 20 from the front quarter view showing front region 28, buttock cheek pads 6, anal region 8 in the form of a central ridge on anal region 8. The central ridge formed by anal region 8 can have a maximum distance from the perimeter of BI pad 10 when viewed from the side (distance 98 in FIG. 11C) in the range of 1-6 cm, preferably 2-4 cm. Furthermore, adhesive 68 can be positioned at multiple places to keep pad 20 properly positioned and adhered to the skin of the user, including to the buttock cheeks and tail bone area.

FIG. 11B illustrates a perspective view of pad 20 from the back quarter view showing front region 28, buttock cheek pads 6, and anal region 8. Furthermore, adhesive 68 can be positioned at multiple places, such as along the perimeter of buttock cheek pad 6 and the waistband region to keep pad 20 properly positioned and adhered to the underwear, thereby avoiding the need for applying adhesives to a skin surface. Additionally, adhesive can be used to form the ridge of anal region 8 by adhering both back surfaces of otherwise flat buttock cheek pads 6 to each other high up in the ridge of anal region 8, for example adhesive 68′ in FIG. 11B. Adhesive 68′ can be applied 1-6 cm, preferably 2-4 cm off of each side of the midline to form a ridge of the desired distance 98.

FIG. 11C illustrates a side view and FIG. 11D illustrates a top view of pad 20 showing how the pad simultaneously conforms to concave up ventral-dorsal curve 2 and convex up gluteal cleft curve 4. In addition or instead of adhesive 68, pad 20 can also be kept in place using straps as described below.

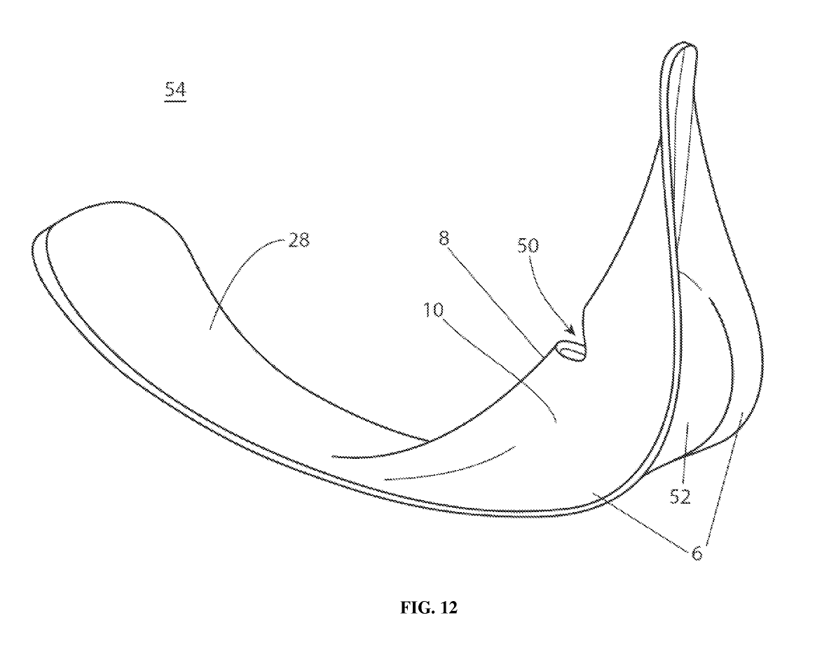

In yet a further embodiment, FIG. 12 illustrates pouched pad 54 which incorporates anal opening 50 in anal region 8 and bowel discharge pouch 52 between buttock cheek pads 6, while maintaining the geometry of pad 20 in FIGS. 11A-D. Anal opening 50 is kept in close proximity to the anus due to the conformance of pad 54 to the anatomy of the user.

Multiple methods are envisioned to maintain the absorbent article in close proximity to the anus. The first method uses dorsal strap 12 and ventral strap 14 (shown cut off in FIG. 1) that provide dorsal force 16 and ventral force 18 respectively. The result of these two force vectors is normal force 22 that maintains anal region 8 of BI pad 10 in contact with the anus. Separately, buttock cheek pads 6 are biased in the open (lay flat) configuration so that they apply gentle pressure of up to 100 grams of force, preferably 50 grams or less, and more preferably 25 grams of force or less on the buttock cheeks throughout the full range of motion ranging from when the user is standing (buttock cheek pads 6 folded on each other) to when the user is sitting (buttock cheek pads 6 lay flat).

With reference for FIGS. 1-6, when laid out flat, the maximum width of BI pad 10 should be in the range of 2-15 times, (preferably 4-8 times) the width of the thinnest portion of dorsal strap 12 to enable proper coverage of the buttock cheeks while ensuring that dorsal strap 12 is not too wide to fit fully in the gluteal cleft up to the tail bone region. If dorsal strap 12 is too wide, it will rest on the buttocks themselves and not fit into the gluteal cleft, which will cause BI pad 10 to not be adjacent to the anus. The typical narrowest width of dorsal strap 12 is in the range of 1 to 2.5 cm, whereas the widest width of BI pad 10 is in the range of 5 to 15 cm.

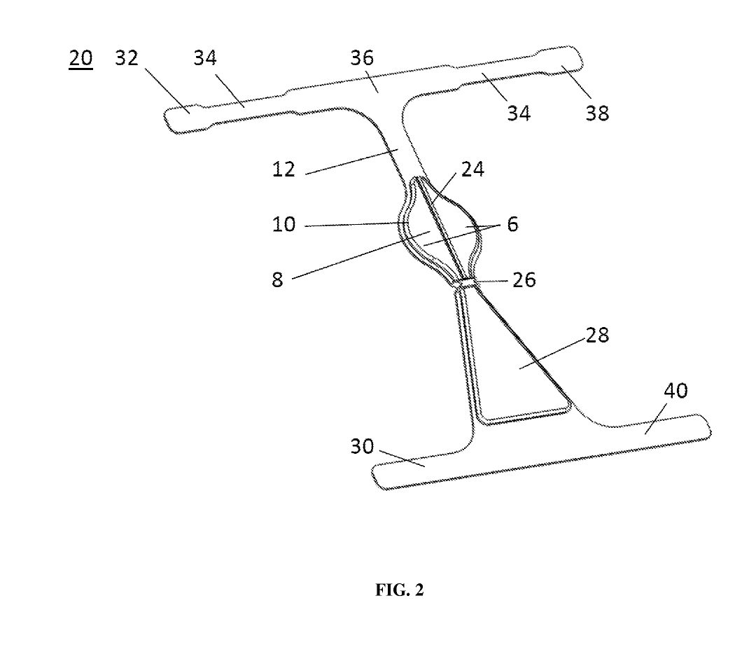

Based on research conducted by the inventors, 70% of people suffering from BI also suffer from urinary incontinence (UI). BI sufferers might also have a need to absorb menstrual discharges using a menses pad. For such users, a pad that can handle both BI and genital discharges is warranted. For example, FIG. 2 illustrates strapped female incontinence pad 20 comprising BI pad 10 described above, together with female genital region pad 28 designed to collect urine or menses, dorsal strap 12, ventral strap 14, rear waist strap 32, right rear tab 32, left rear tab 38, front right waist strap 30 and left front waist strap 40.

To use female incontinence pad 20, the user inserts it between her legs, pulls both straps to position BI pad 10 adjacent to the anus and then secures rear strap 36 and front straps 30 and 40 around the waistline. Rear strap 36 can have elastic region(s) 34 to enable a fit of various sized users. Right rear tab 32 and left rear tab 38 can releasably attach to right front strap 30 and left front strap 40 respectively at any point along the straps to further enable the fit of various sized users.

To dispose of pad 20, the user releases tabs 32 and 38 from straps 30 and 40, removes soiled pad 20 from between her legs, folds and discards. The packaging or wrapper of a new pad 20 can be used to discard the soiled pad 20 in a clean and hygienic manner. The packaging or wrapper can be scented or incorporate odor control technology to neutralize the odor associated with the disposal of a used pad.

With respect to FIG. 2, BI pad 10 has fold 24 that enable a lay flat pad 10 to conform to the complex surface formed by curves 2 and 4. Many such folds 24 can be incorporated to pad 10. Alternative and equivalent structures to folds 24 can be pleats, creases, grooves, corrugations, areas of elasticity or areas of thinness or weakness incorporated along one of more axes, at least a portion of the perimeter, radials or chords of BI pad 10.

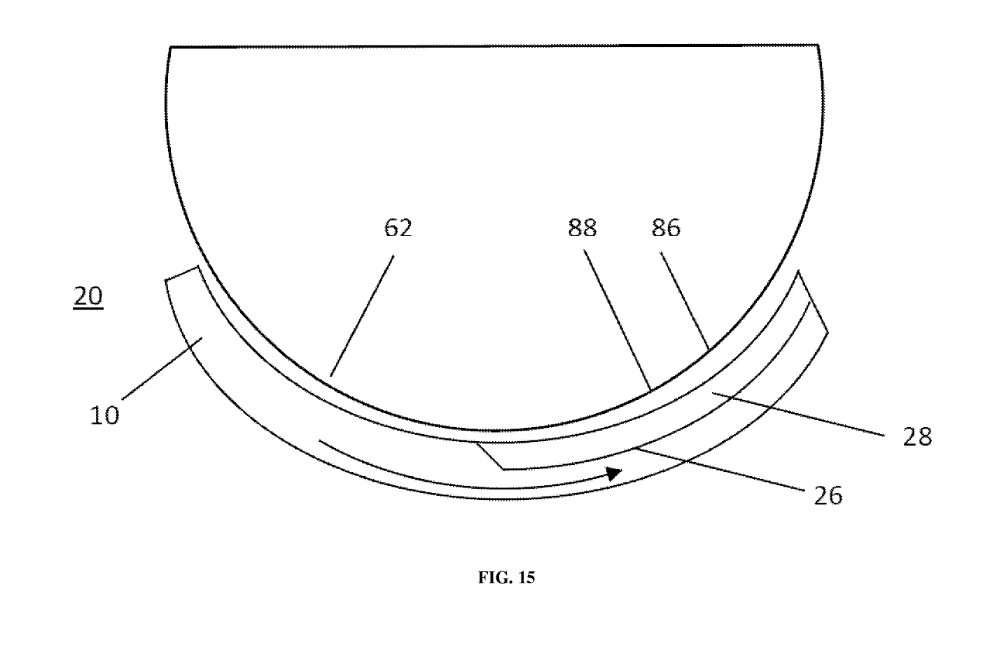

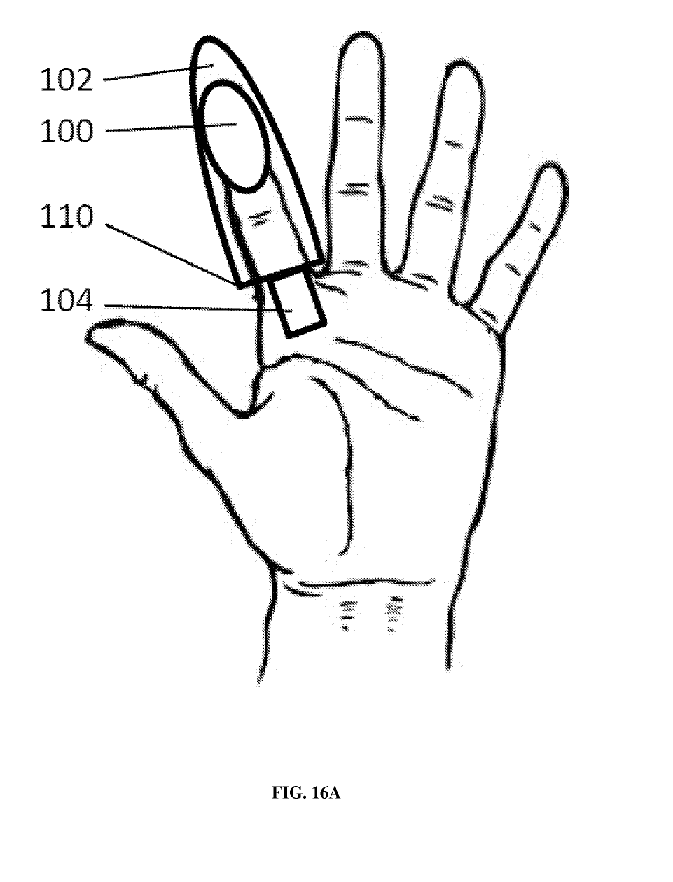

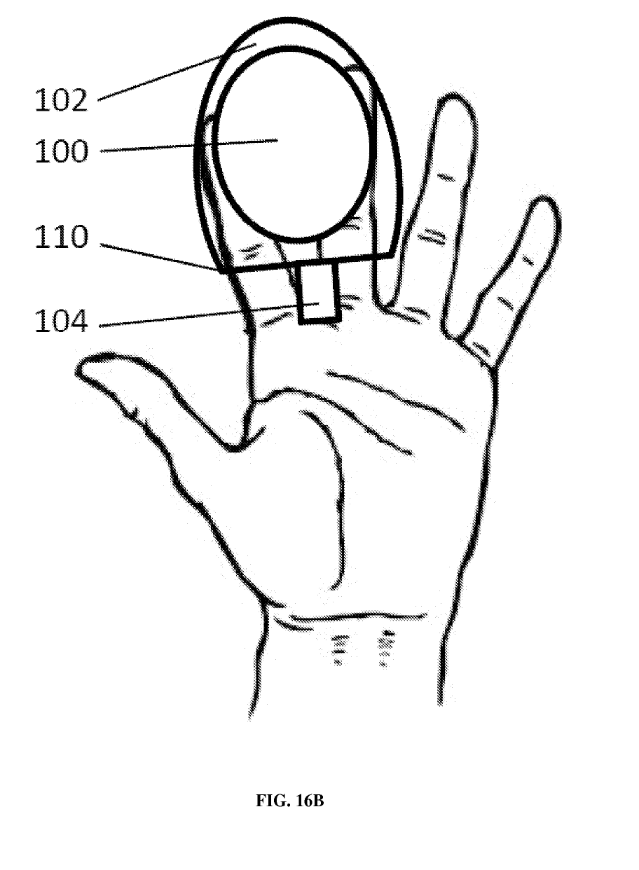

A further feature of the present invention is barrier 26 illustrated in FIG. 2 that prevents bowel discharge fluid on BI pad 10 from flowing into female genital region pad 28 in order to prevent infections by bowel matter, such as yeast infections. Barrier 26 also prevents urine or menses from flowing back and using the absorption capacity of BI pad 10 reserved for bowel discharge. In so doing, urine or menses don't wet and irritate the buttock cheeks and perianal region. Barrier 26 can be a hydrophobic element, a crushing or melting of the pad material itself, a physical dam, a notch, or a gap in the two pads. Pad 10 and region 28 can be one pad physically delineated into two regions by barrier 26. Barrier 26 can also be a fluid-impervious layer between BI pad 10 and genital region pad 28 that overlap each other at least for some length allowing for stacked, two-layer absorption without cross talk. In this manner, bowel discharge from anus 62 can be channeled down along BI pad 10 due to physical features (for example grooves, channels, or corrugations) or wicking design (for example fiber alignment) in the direction of the arrow in FIG. 15 and underneath genital region pad 28. Barrier 26 prevents cross contamination of the BI and genital discharges. In this embodiment, at least some portion of BI pad 10 and genital region pad 28 overlap in a fused (i.e. inseparable) two-layered structure with BI pad 10 external to genital region pad 28 for at least some portion of pad 20 and separated by barrier 26. The advantages of this embodiment include better odor control since the BI discharge is pulled into and trapped between two impervious layers (barrier 26 and the outer layer of pad 20), improved desiccation of the BI discharge due to increased area of BI pad 10 which now extends over at least a portion of the genital region, genital discharges from vagina 86 or urethra 86 do not wet or contaminate BI pad 10, and BI discharge does not contaminate the genitals or genital region pad 28 of pad 20. In such an embodiment, each side of the pad could have a unique label, marking, shape or color to assure proper orientation by the user. For example, at least a portion of BI pad 10 could be blue and at least a portion of genital region pad 28 could be pink.

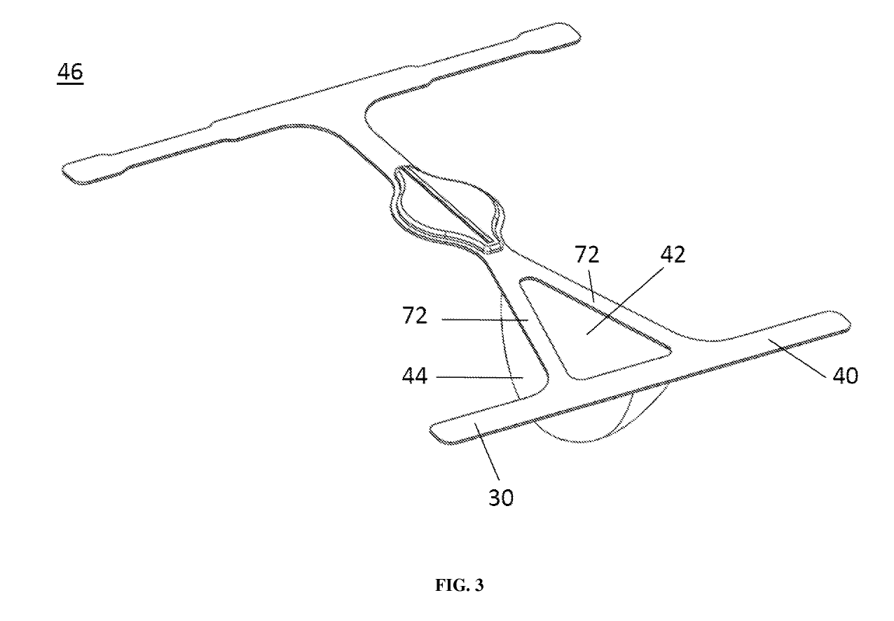

FIG. 3 illustrates a further embodiment of the present invention directed at male suffers of BI and UI. The embodiment of FIG. 2 is similar to that of FIG. 3 with the exception that strapped male pad 46 incorporates two genital flanking straps 72 that surround front genital opening 42 and male genital pouch 44 to enable urine to be caught in the absorbent material within or integrated into pouch 44. Urine could also be sealed and confined by a seal around the genital area defined by the perimeter of front genital opening 42. Strapped male pad 46 can also incorporate barrier 26 (not shown) to prevent urine from soaking or wicking into BI pad 10 and is worn and removed in a manner similar to strapped female pad 20.

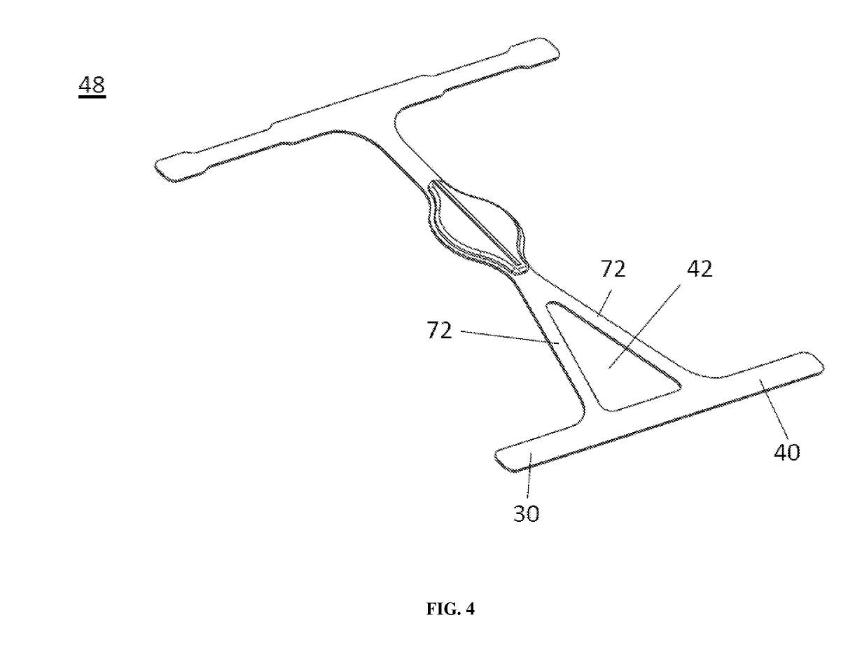

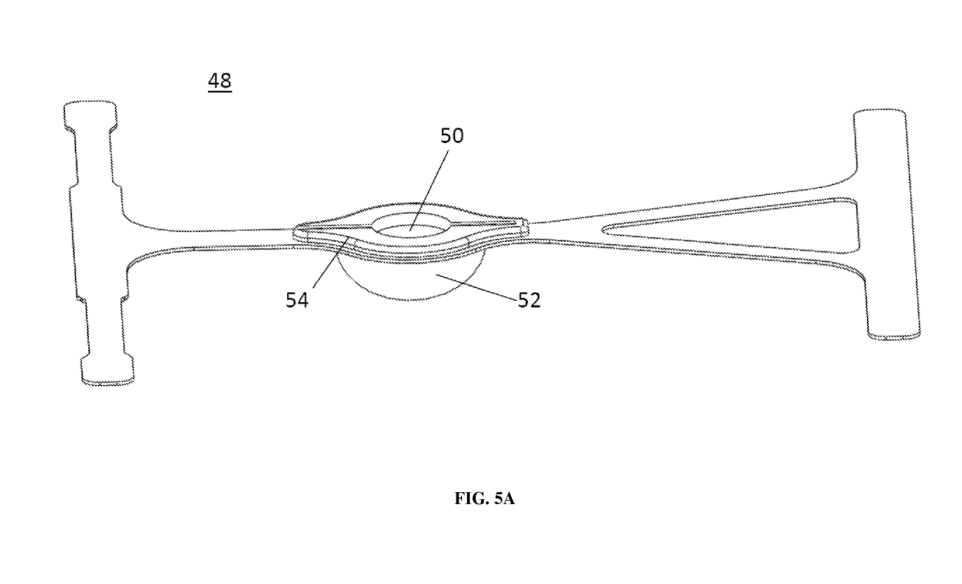

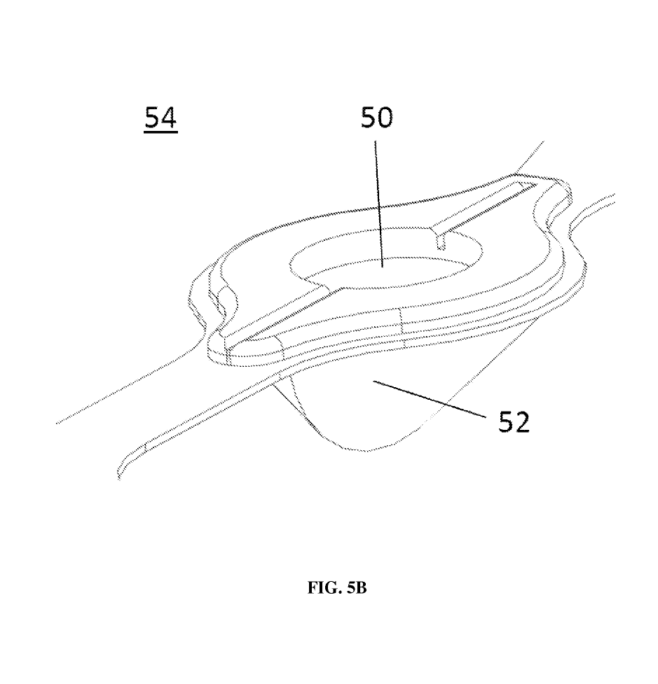

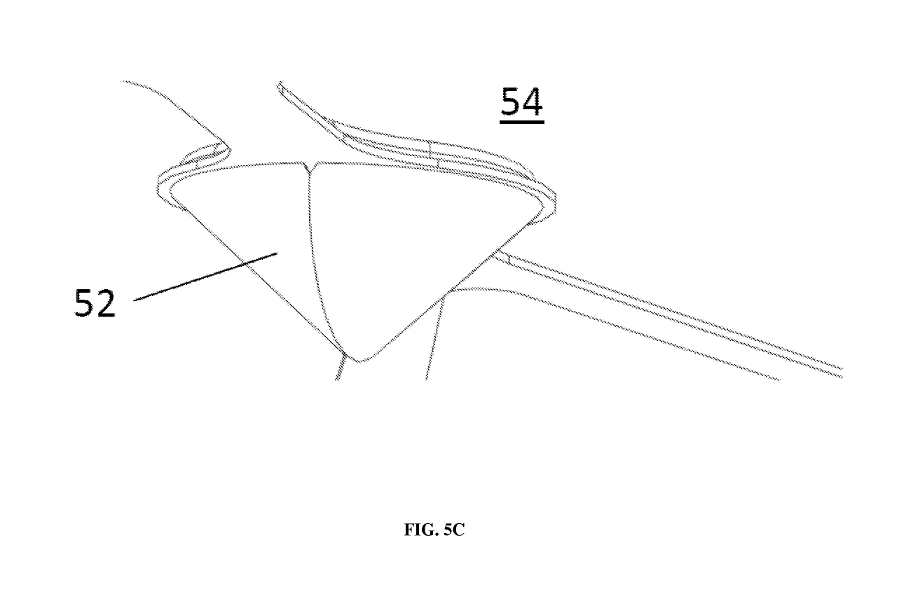

In yet a further embodiment of the present invention, FIG. 4 illustrates strapped BI pad 48 that can be worn by either male or female users with front genital opening 42 formed by genital flanking straps 72 and front straps 30 and 40. Pad 48 is for users that don't have a need for UI or menses protection. Alternatively, pad 48 can be complemented by a user wearing a traditional UI or menses protection pad, normally adhered to their undergarments, since front genital opening 42 flanks the genitals and does not interfere with the flow of either urine or menses.

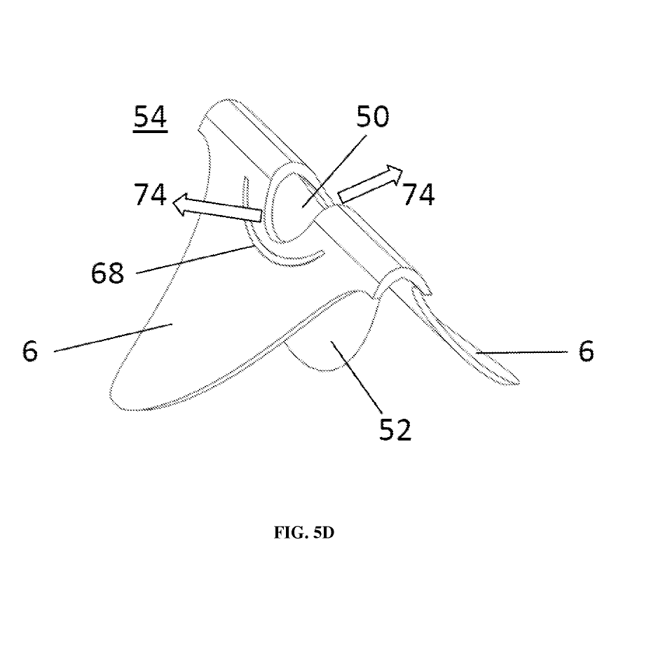

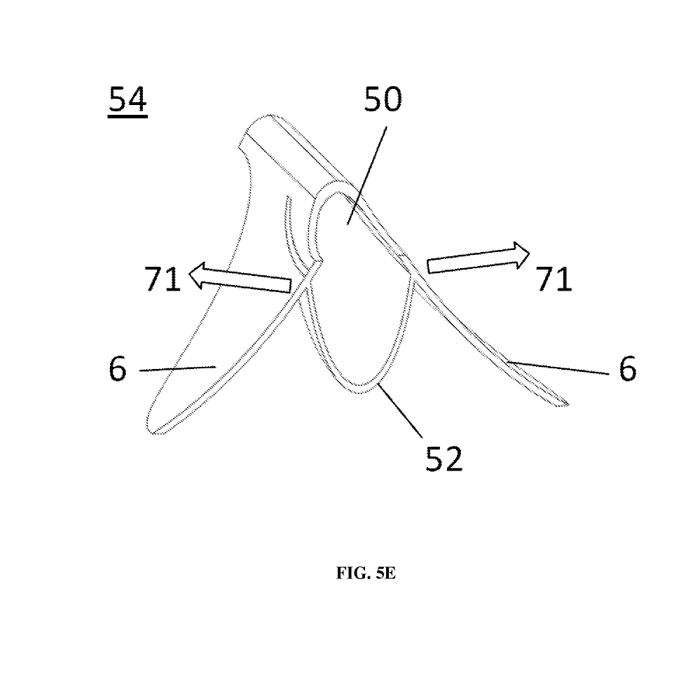

FIGS. 5A-E illustrate yet a further embodiment of the present invention. Pouched BI pad 54 is similar to BI pad 10 with the addition of anal region opening 50 and bowel discharge pouch 52. Pouched BI pad 54 is intended for the capture and containment of liquid, mucosal and solid stool that is greater than the absorption capacity of the material from which pouched BI pad 54 is made. FIG. 5A shows pouched BI pad 54 incorporated into strapped BI pad 48. FIGS. 5B-C illustrate a close up of pouched BI pad 54, anal opening 50 and bowel discharge pouch 52 from above (FIG. 5B) and below (FIG. 5C). Bowel discharge pouch 52 can consist of pleats, folds or otherwise configured to be able to fold flat and also open into a surface that defines a pouch space.

FIG. 5D illustrates pouched BI pad 54 folded as it would fit between the buttock cheeks in the gluteal cleft. Anal region opening 50 can be laterally stiff enough to supply at least 100 grams of radial opening force 74 to keep opening 50 from deforming into a narrow slit between the buttocks cheeks. Opening 50 should maintain a circular or elliptical shape even when the user is standing and the buttock cheeks are compressed together so that any bowel discharge will pass through opening 50.

FIG. 5E is a cut-away view of pouched BI pad 54 of FIG. 5D showing how the elasticity of the folded material that makes up bowel discharge pouch 52 can provide lateral force 71 of up to 100 grams to keep anal region opening 50 in an elliptical shape and buttock cheek pads 6 up against the buttock cheeks throughout their normal range of motion. FIG. 5E further illustrates that opening 50 is smaller than the width and length of pouch 52, thereby ensuring that bowel discharge becomes trapped in pouch 52 and does not come in contact with the user's skin. Bowel discharge pouch 52 is either empty or filled with a wicking material to move fluid away from opening 50, or alternatively filled with highly fluid absorbent material known in the art of diaper construction, such as polyacrylic acid.

The material from which BI pad 10, BI pad 54 and pouch 52 are made can be absorbent on the skin contacting side (using materials utilized in urinary incontinence, feminine hygiene pads, BI pads or baby diapers) with a fluid impervious coating on the outside. Alternatively, for increased comfort, the material can be fully or partially absorbent on both sides with a fluid impervious layer in between. A portion of BI pad 10, BI pad 54 and pouch 52 can also incorporate an odor control material such as activated charcoal and the like, as is known in the art of diaper and pad construction.

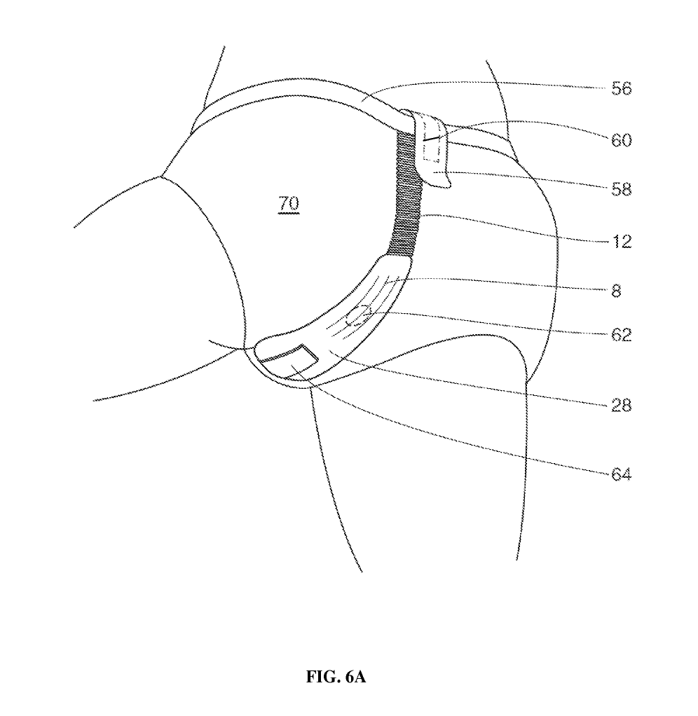

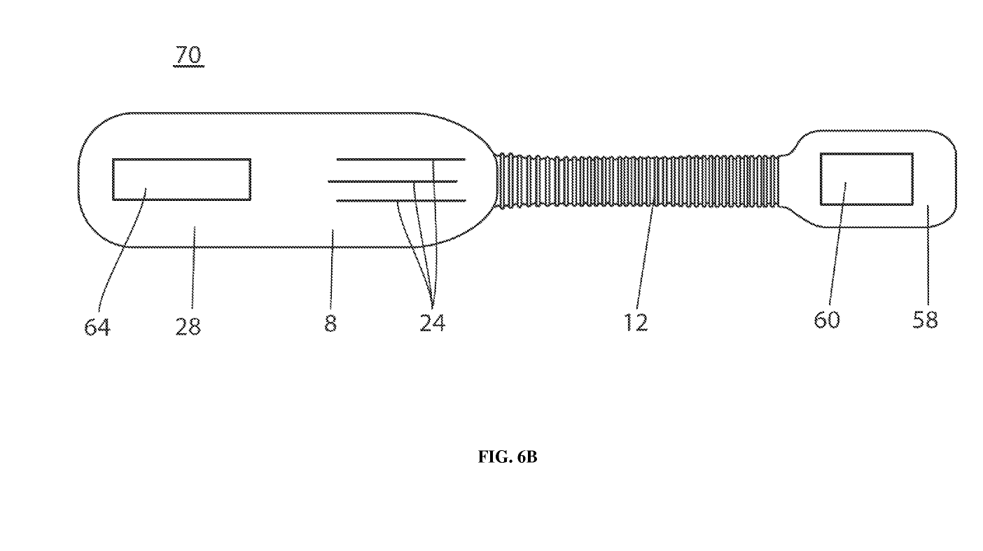

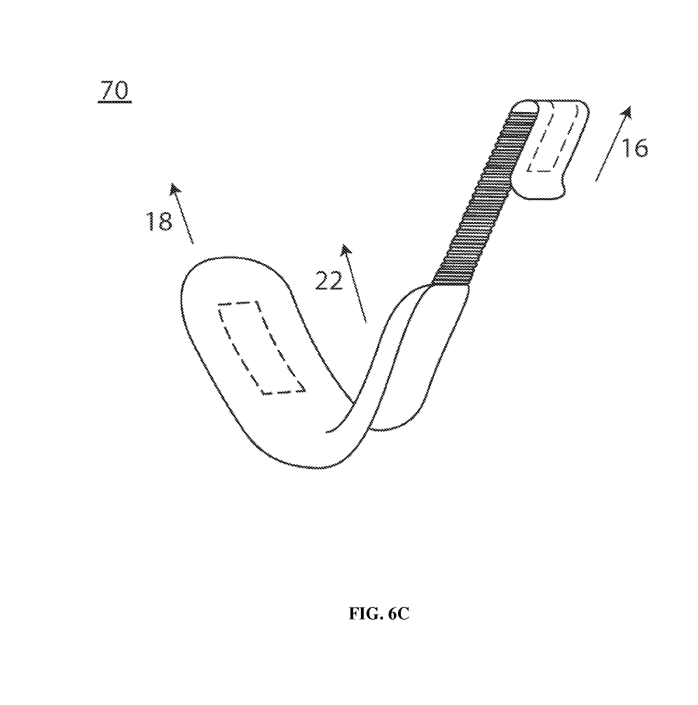

FIGS. 6A-C illustrate yet a further embodiment of the present invention. In FIG. 6A, BI pad 70 is shown being worn by a user through transparent underwear. As further illustrated by FIG. 6B, genital region pad 28 having front attachment region 64 of BI pad 70 is adhered to the user's underwear much like traditional feminine hygiene pads. Anal region 8 incorporates folds 24 that allow BI pad 70 to conform to the complex curves 20 and 4 as defined in FIG. 1. Dorsal strap 12 is elastic and is pulled up along the tailbone of the user so that dorsal force 16 which is countered by ventral force 18 creates normal force 22 that keep anal region 8 in contact with anus 62, as illustrated in FIGS. 6A and 6C. Dorsal strap 12 can maintain a dorsal force 16 of up to 500 grams to keep anal region 8 in contact with anus 62.

Rear strap tab 58 incorporates an attachment region 60 that is attachable to the user's skin in the region of the tailbone, or to waistband 56 using adhesive, buttons, or hook and loop mechanisms for example. Waistband 56 can be part of a normal pair of underwear, or alternatively a stand-alone waistband to which dorsal strap 12 of BI pad 70 can be attached. An alternative embodiment would comprise BI pad 70 having both ventral strap 14 and dorsal strap 12 that both attach to waistband 56.

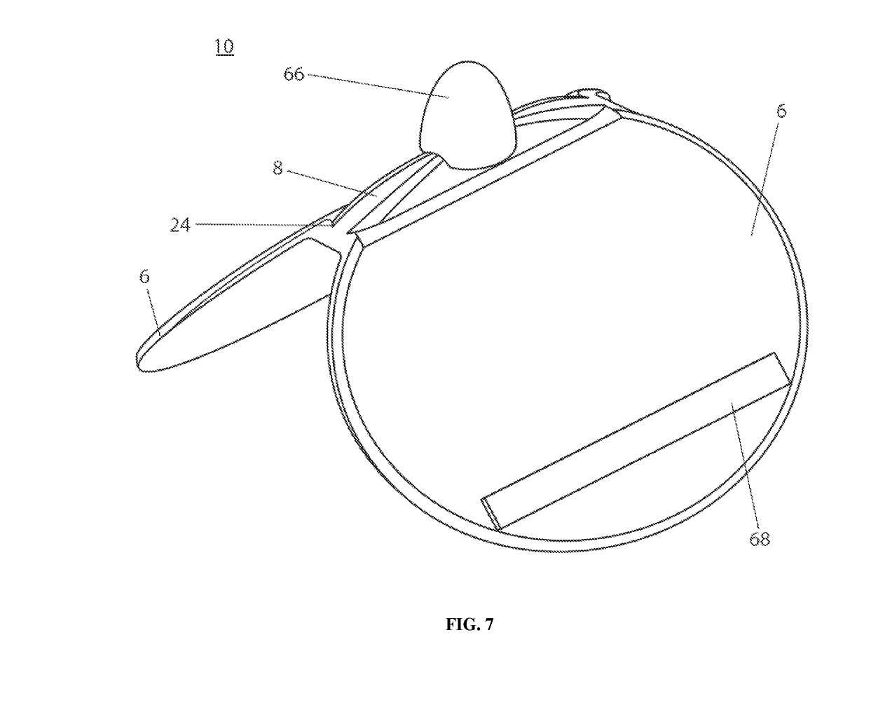

FIG. 7 illustrates a further embodiment of the present invention. In this embodiment, BI pad 10 incorporates anal penetrating element 66 that rests against or enters into the anus. Anal penetrating element 66 can enter anywhere from 0 to 4 cm into the anal canal and be in the range of 0.3 cm to 1.5 cm in diameter, more preferably 0.5 to 1 cm in diameter. Element 66 can simultaneously serve one or more of the following functions:
i. block the flow of stool through the anal canal,

ii. absorb and contain liquid stool,

iii. channel the flow of stool into a collection pouch, and/or

iv. wick liquid out to an absorbent pad or collection pouch.

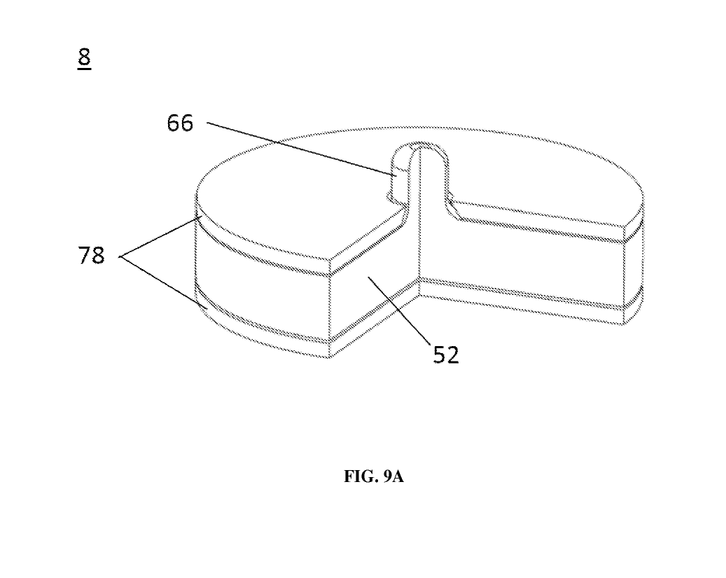

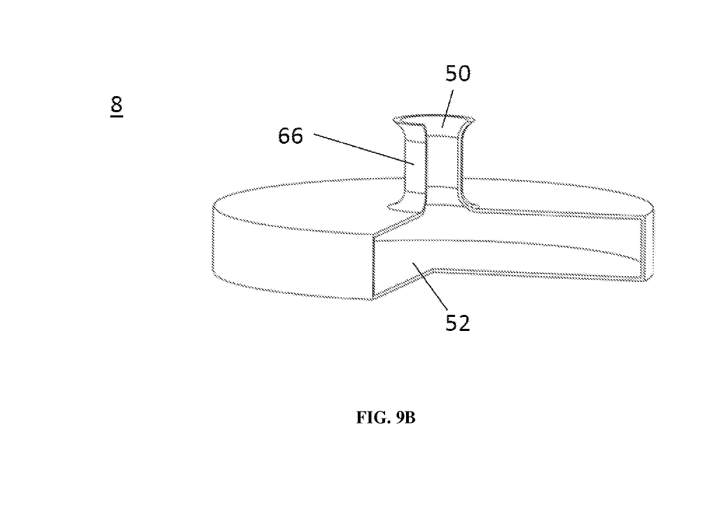

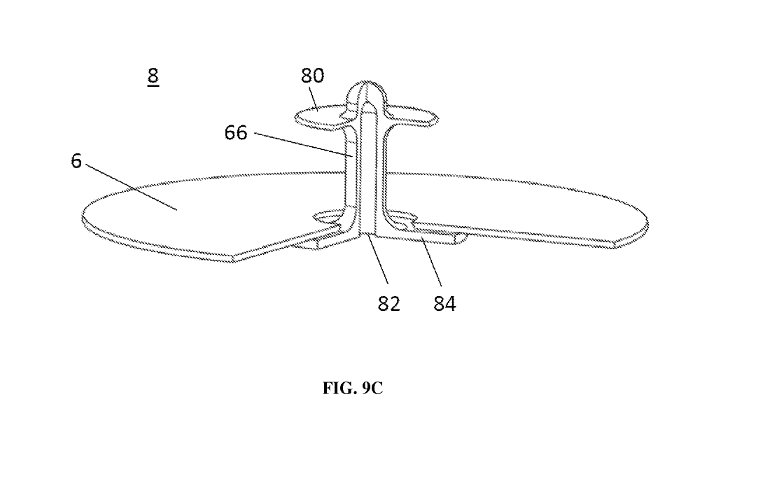

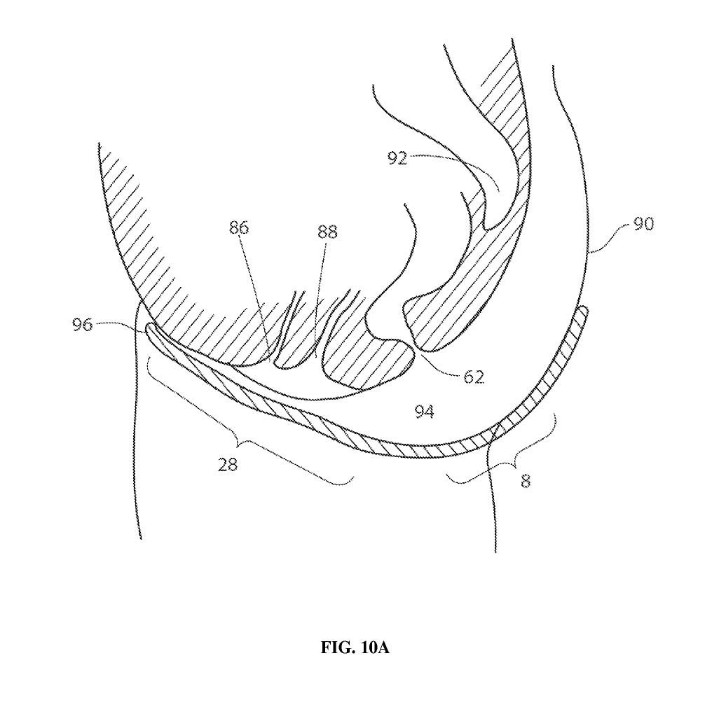

For example, element 66 can be used to enhance absorption of bowel discharge right at the anus and to wick it to an area of BI pad 10 that has sufficient absorption capacity. Buttock cheek pad 6 in the embodiment illustrated in FIG. 7 can facilitate absorption in an internal layer or space and be coated with fluid impervious layers 78 (FIG. 9A) on both sides so that the absorption can occur and be trapped in buttock cheek pad 6 without the fluid coming into contact with the button cheek skin. Alternatively, anal region 8 can be narrow and through wicking action alone, channel liquid bowel discharges to a coated reservoir in the direction of the tail bone.

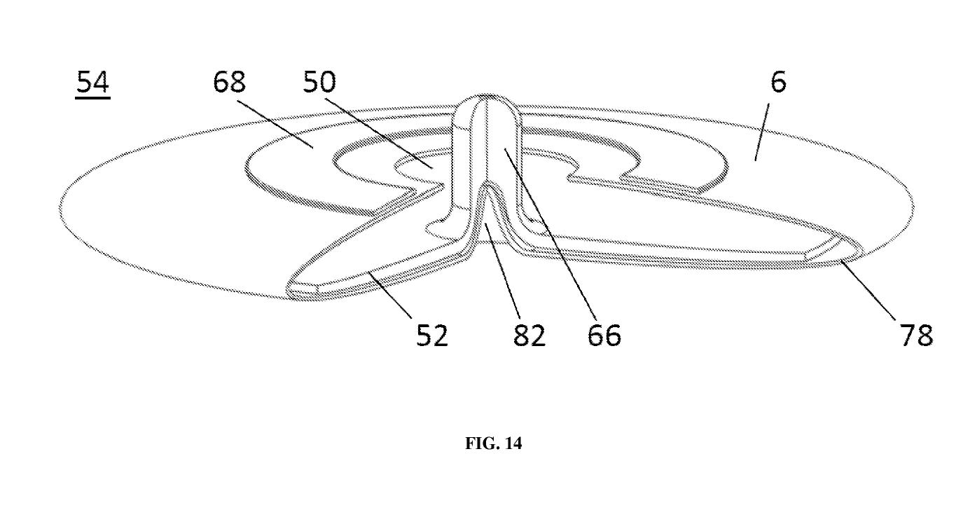

Element 66 can be roughly the size of a cotton ball and of similar consistency. Alternatively, element 66 cannot penetrate into the anal canal itself, but rather be an elongated shape, such as a semi-circular or circular central ridge, of approximately 0.5-2 cm thickness, preferably between 0.75-1.5 cm thickness and between 1-6 cm long, preferably between 2-4 cm long, along the top of the gluteal cleft in anal region 8 to form a “pillow top” pad with high porosity so that fluid will be contained in this ridge, or wicked into the smaller pores of the absorbent pad away from the ridge and the user's skin. Element 66 can be pushed into the anus using a finger from the bottom side of BI pad 10 using concavity 82 (FIGS. 9C and 14), helping to position BI pad 10 in the right location. BI pad 10 with element 66 can be part of strapped pads 20, 46, 48 and 70.

FIGS. 9A-C illustrate further embodiments of anal penetrating element 66. In FIG. 9A, element 66 is a porous element that wicks moisture into bowel discharge pouch 52 which is either empty or filled with an absorbing material. Bowel discharge pouch is surrounded on one or both sides by fluid impervious layer 78.

In FIG. 9B, element 66 is a non-absorbent and hollow collection tube with or without a flared upper end of anal region opening 50 that channels stools into bowel discharge pouch 52, shown here as an empty collapsible bladder.

In FIG. 9C, element 66 in an anal insert or anal plug with our without top disk 80 that blocks stool from passing through the anal canal. Bottom disk 84 can be connected to buttock cheek pad 6 to catch any material that leaks around element 66. Concavity 82 can receive within it an applicator or fingertip for inserting element 66 into the anus.

Various permutations and combinations of anal penetrating element 66 are also envisioned. Examples are an absorbent element surrounded by a non-absorbent collection tube, or a collection tube with upper disk 80 for improved anchoring.

Anal penetrating element would preferably be in the range of 0.3 cm to 1.5 cm in diameter, more preferably 0.5 to 1 cm in diameter.

In all embodiments described herein, anal penetrating element 66 should be soft in order to be comfortable. Example softness is in the range of a cotton ball to felt for an absorbent element 66, and shore 40A silicone of softer for non-absorbent elements 66.

In a further embodiment, BI pad can be secured in place not with a strap, but rather with the use of skin adhesive elements 68 that can be present on any part of buttock cheek pad 6. Skin adhesive element 68 can be the entire size of buttock cheek pad 6 and adheres to the skin (e.g. polyurethane, polyethylene or polypropylene films in the thickness range of 0.00025″ to 0.004″, preferably 0.0005″ to 0.002″ thick) with low tack acrylic adhesives or incision films used in surgery to cover the skin before and during a surgical procedure). The film would be highly elastic and easy to apply in the gluteal cleft by, for example, simply sitting on it. The film can contain an absorbent pad in the area of the anus.

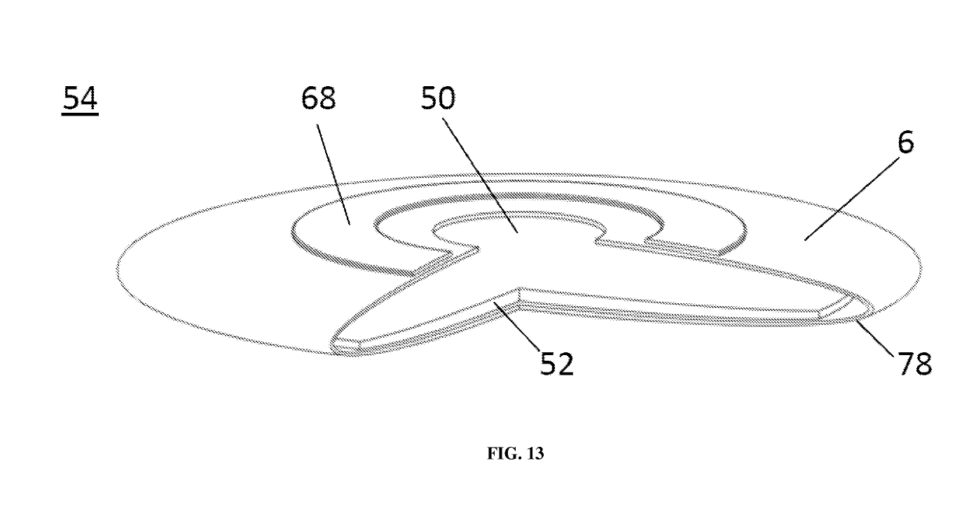

In a further embodiment, as illustrated in partial cutaway view in FIG. 13, pouched BI pad 54 can be made from film 72 which forms a bladder with anal region opening 50 in the area of the anus surrounded by adhesive 68 in a concentric ring taking 1 to 100% of the available upper surface of pouched pad 54, and absorbent material inside bowel discharge pouch 52 that would absorb the discharge coming from the anus through the hole in the film. BI pad 10 in this embodiment can be applied by removing the protective layer off of adhesive 68 and simply sitting on it, or pushing it folded into the gluteal cleft to allow adhesive 68 to come into contact with the skin and seal the perimeter of anal opening 50. Pouched pad 54 can have a peel off tab without adhesive to enable easy removal of a used pad.

In yet a further embodiment illustrated in FIG. 14, PI pouched pad 54 incorporates anal penetration element 66 (in all its permutations) inside anal opening 50. In the embodiment shown in FIG. 14, anal penetrating element 66 is in fluid communication with absorbent material inside bowel discharge pouch 52.

Pouched BI pad is shown as a circular shape in plan view in FIGS. 13 and 14, but can also be butterfly-wing shaped or two ellipses that connect along a chord or tangent point similar to the embodiment in FIG. 7.

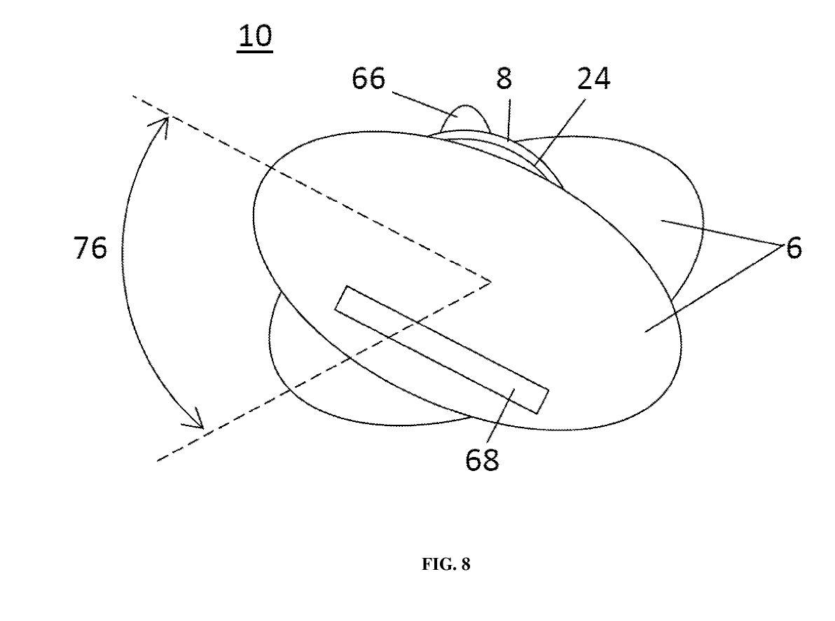

FIG. 8 shows a further embodiment wherein the buttock cheek pads 6 can move laterally with respect to one another by up to 2 cm, or pivot so that their longitudinal axes form angle 76 relative to each other to accommodate relative movement of the buttocks cheeks during walking, running, etc. Angle 76 can be up to 90 degrees, preferably up to 45 degrees. Folds 24, or the elastic properties of anal region 8, can enable the relative motion described above.

In yet a further embodiment related to FIGS. 2-5, rear strap 36 can be pre-attached to front straps 30 and 40 and require the user pull on the strapped pad like conventional underwear. Once worn, the straps can be separated to remove the soiled pad in an open manner between the legs, or alternatively pulled off like normal underwear. Adjustment of one or more of the straps (e.g. dorsal strap 12) can be done after pulling on the pad like a pair or underwear. Alternatively, the pad can be made in various sizes or with sufficient elasticity to fit a broad range of user sizes without adjustment.

In a yet further embodiment, BI pad 10 is adhered to an undergarment specifically designed to keep anal region 8 next to the anus. This undergarment is designed with a highly elastic rear portion that has pre-shaped regions for the buttocks that fits up against dorsal-ventral curve 2 to keep BI pad 10 positioned there.

In a yet further embodiment, BI pad 10 is kept in place by wearing a thong-like pair of underwear wherein the back strap of the thong runs up the back side of the ridge defined by anal region 8 and keeps in next to the anus.

In yet a further embodiment as depicted in FIG. 9C, BI pad 10 can be kept in place by making it the bottom disc of an anal plug or anal insert (see for example WO 2009/060437). An anal plug where the bottom disk comprises a pad would capture any leaked anal discharge. The anal plug would keep the pad in place up against the anus.

In a yet further embodiment, pad 10 can be used for purposes other than bowel incontinence. For example, at least some portion of pad 10 can incorporate impregnated within it or on top of it an agent or drug that is intended for close contact with perianal skin, external anus, or even inside the anus or anal canal (see anal penetrating element 66 in FIG. 7). Examples agents and drugs include anti-inflammation, anti-pain, anti-itch, anti-hemorrhoid, zinc-oxide, or anti-fissure creams or lotions. Pad 10 can form an occlusive bandage that keeps an agent or drug in contact with the skin for extended periods of time.

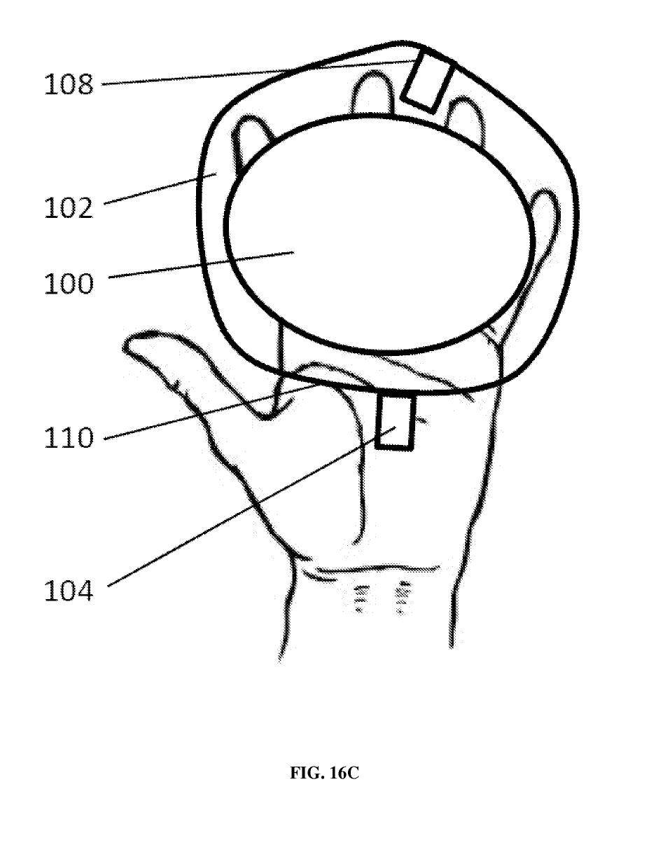

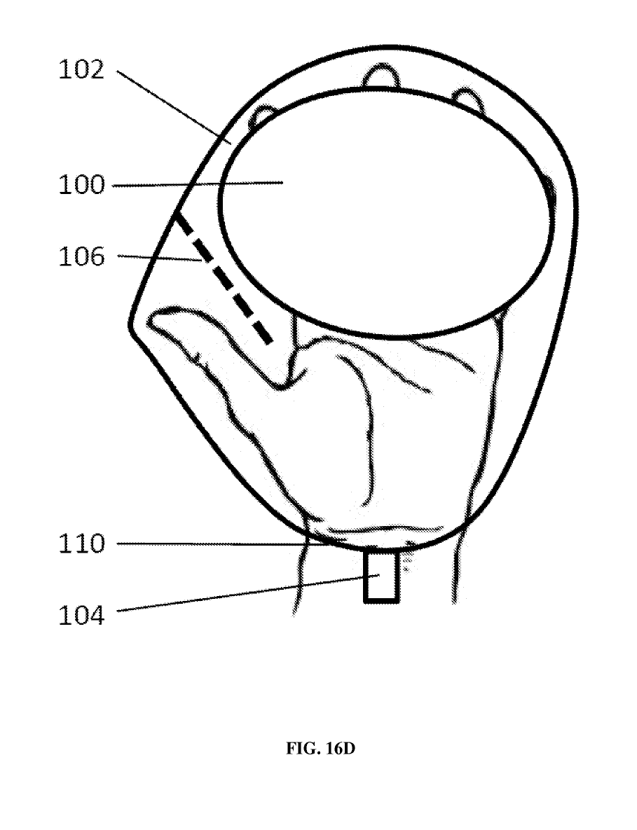

In yet a further embodiment, absorbent article 100 can be mounted on the palm side of a fluid-impermeable (e.g. polyethylene) hollow cot 102 as pictured in FIG. 16 a-d. Cot 102 can completely cover a finger (FIG. 16a), two fingers (FIG. 16b), four fingers (FIG. 16c), or an entire hand (FIG. 16d). Absorbent article 100 can be a dry or wet wipe for cleaning the perianal skin region. In a further embodiment, absorbent article 100 can be pre-impregnated with or have applied to it an agent and/or drug (for example anti-inflammation, anti-pain, anti-itch, anti-hemorrhoid, zinc-oxide, or anti-fissure creams or lotions). In this embodiment, the wipe, agent or drug is applied without risk of contaminating the applying hand by contact with the perianal region. Optional elastic back region 110 serves to seal around cot 102 around the finger(s) or hand. Back tab 104 can be used to remove cot 102 with the opposite hand while internal seam, weld or cleft 106 and/or internal tab 108 can be grasped by the fingertips from within cot 102 while pulling back tab 104 forward towards the fingertips to completely invert cot 102 so that contaminated absorbent article 100 is fully contained within the interior of inverted cot 102. The inverted surface of cot 102 remains clean and can be handled without contacting contaminated absorbent article 100 until cot 102 is disposed of. Cot 102 can be made of two sheets of a thin impervious material welded along its periphery or alternatively a section of lay flat tubing can be cut and welded to size.

In a further embodiment, absorbent article 100 detaches from cot 102 due to adhesive or the tackiness of an agent or cream applied to absorbent article 100 which comes into contact with the perianal skin and removes absorbent article 100 from its impervious cot 102 backing. In this manner, absorbent article 100 can be applied to the perianal region without contaminating the applying hand.

In a further embodiment, cot 102 can be single sided and has straps, tabs or finger holds on the side opposite absorbent article 100 to reversible attach cot 102 to the applying hand. In a further embodiment, absorbent article 100 when impregnated with an agent or drug can be covered by a protective and impermeable plastic layer that is removed before use.

As used herein the term “about” refers to ±10%.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following example.

We claim:

1. An absorbent article comprising an anal pad configured for folding along an axial center line to fit between buttock cheeks, said anal pad being configured for wicking moisture away from an anus to an absorbent material positioned against said buttock cheeks and/or at a genital region.

2. The absorbent article of claim 1, wherein said anal pad includes a first pad region for contacting said anus and a second pad region including said absorbent material.

3. The absorbent article of claim 1, wherein said anal pad is held in position up against the anus using an adhesive and/or a ventral strap.

4. The absorbent article of claim 1, further comprising a genital pad not being in fluid communication with said anal pad.

5. The absorbent article of claim 1, wherein said anal pad is configured for occupying a gluteal cleft space.

6. The absorbent article of claim 2, wherein said anal pad includes a central ridge being offset at least 2 cm from a plane of a perimeter of said anal pad when said anal pad is positioned within said gluteal cleft space.

7. The absorbent article of claim 6, wherein said first pad region is positioned at said central ridge and said second pad region is positioned along said perimeter.

8. The absorbent article of claim 4, wherein said anal pad overlaps at least a portion of said genital pad.

9. The absorbent article of claim 1, wherein said anal pad can assume a complex curve with a maximum radius of 10 cm along at least two axes while retaining conformance to the anatomy.

10. An absorbent article comprising an anal pad configured for wicking moisture away from an anus to an absorbent material positioned against buttocks or buttock cheeks and a genital pad not being in fluid communication with said anal pad.

11. The absorbent article of claim 10, wherein said anal pad includes a first pad region for contacting said anus and a second pad region including said absorbent material.

12. The absorbent article of claim 10, wherein said anal pad is configured for occupying a gluteal cleft space.

13. The absorbent article of claim 12, wherein said anal pad includes a central ridge being offset at least 2 cm from a plane of a perimeter of said anal pad when said anal pad is positioned within said gluteal cleft space.

14. The absorbent article of claim 13, wherein said first pad region is positioned at said central ridge and said second pad region is positioned along said perimeter.

15. The absorbent article of claim 10, wherein said anal pad overlaps at least a portion of said genital pad.

16. The absorbent article of claim 10, wherein said anal pad can assume a complex curve with a maximum radius of 10 cm along at least two axes while retaining conformance to the anatomy.
